(19) United States
(12) Patent Application Publication
(10) Pub. No.: US 2009/0047740 A1
(43) Pub. Date: Feb. 19, 2009

(54) CULTURE AID FOR CELLS AND TISSUES

(75) Inventors: Jennifer H. Elisseeff, Baltimore, MD (US); Christopher Williams, Baltimore, MD (US); Ilksen Gurkan, Stilwell, OK (US); Shyni Varghes, San Diego, CA (US)

Correspondence Address: BELL, BOYD, & LLOYD LLP, P.O. BOX 1135, CHICAGO, IL, 60690, US

(73) Assignee: Johns Hopkins University, Baltimore, MD (US)

(21) Appl. No.: 11/612,133

(22) Filed: Dec. 18, 2006

Related U.S. Application Data
(63) Continuation of application No. PCT/US2005/020707, filed on Jun. 9, 2005.
(60) Provisional application No. 60/583,588, filed on Jun. 28, 2004.
(60) Provisional application No. 60/578,627, filed on Jun. 10, 2004.

(51) Int. Cl. C12N 5/02

(52) U.S. Cl. 435/396

(57) ABSTRACT

A biological culture medium provides a three-dimensional framework for cell growth. The medium comprises a film and a matrix.

### BACKGROUND OF THE INVENTION

Simulating the in vivo environment for the culture of cells and tissues can be essential to ensure and to obtain growth and development of cells as well as development of tissues and organs in vivo. A three-dimensional representation of the environment in which a cell exists in vivo may be essential to ensure proper differentiation and function of that cell in vitro. Films and hydrogels may provide such simulation for the more realistic growth of cells and tissues in vitro.

Cross-linked polymeric biomaterials are being used in biomedical applications including coatings for medical devices, implants, scaffolds and drug delivery vehicles. Polymer networks may be formed, for example, by crosslinking water soluble monomers or polymers to form a water insoluble polymer network. Mechanical and structural properties may be manipulated by modification of the crosslinking density which controls, for example, network pore size, water content and mechanical properties.

Films can be configured to more closely simulate the natural surfaces on which cells grow in vitro, as compared to glass or the various types of plastics found in cultureware. Films enable portability. Films also can be modified to carry reactive, functional groups to enhance and/or to facilitate reaction with cells.

### SUMMARY OF THE INVENTION

The instant invention provides a composition comprising a biologically compatible film and a biologically compatible matrix material to provide a support and scaffold for cell growth and differentiation. The composition can further comprise a polymer functionalized by at least one reactive moiety for binding to the matrix to provide a multilayered culture medium. In some embodiments, the matrix is, for example, a hydrogel. The hydrogel can be one with one or more functional groups that react with functional groups on the film.

In other embodiments, the functionalized polymer comprises at least 10 monomeric units, at least 100 monomeric units or at least 1000 or more units of monomer. The functionalized polymer can comprise at least two reactive moieties, with plural copies of each of said at least two reactive moieties. The at least two reactive moieties can react with different chemical structures on the matrix and on one or more different target entities to provide the functionalized polymer with a predetermined orientation and directed, specific reaction with a target entity.

The reactive moiety may be selected, for example, from methacrylates, ethacrylates, itaconates, acrylamides, thiols, peptides and aldehydes. For example, a polypeptide having a certain electronic configuration or a binding ability can be reactive group if that peptide interacts and binds to a complementary ligand or binding partner on a target surface. Thus, a collagen helix can be a suitable reactive moiety for binding to another collagen helix found in a target entity.

In a polymer of interest, not all monomers need be functionalized with a reactive moiety.

If more than one reactive moiety is present, the functionalized polymer can contain substantially equal molar amounts of the at least two different reactive moieties. When more than two reactive moieties are present, generally, the moieties comprise two classes of molecules that are reactive with two target entities, that is, the moieties of one class, while chemically distinct, react with the film, although, the reaction may be with two different chemical structures on the film, and the other class of reactive moiety(ies) react with another target site, such as a functionalized polymer of interest.

In a functionalized polymer, to ensure directionality, either the backbone bonds of the polymer are flexible to obtain rotation about the axis of the polymer or all of one species of moiety are present on the same side of the polymer or are in the same orientation on the polymer.

The matrix of interest can be, for example, any biologically compatible hydrogel known in the art or made as taught herein. The hydrogel can have one or more species of functional groups, which can be present on a hydrogel reactant, can be added, for example, synthetically or can be incorporated into the hydrogel. The functional groups of the matrix are the same as the reactive moiety of the polymer of interest described hereinabove. The matrix presents a porous, three-dimensional support for relatively free passage of fluids, conduits for cell movement, carrier for active agents and substrates or surfaces to support cell growth. A matrix has the presentation of a sponge, for example.

The film of interest can be any biologically compatible film known in the art or made as taught herein. The film can be biodegradable. For example, films made of hyaluronic acid derivatives, such as Seprafilm, cyanoacrylates, Gore-Tex, Interceed, colloidians, amylpectin derivatives, fibrin derivatives, glucosamine derivatives, hydrogel films, polyethylene glycol derivatives and the like can be used. The film can be naturally occurring, made from naturally occurring components or be synthetic. The film can be treated to carry reactive functional groups, said groups reactive, with, for example, a matrix of interest. The functional groups of the film are the same as those for a functionalized polymer of interest. The film enables location of plural sites for cell growth by the construction of plural, discrete culture sites at different regions of a film. The film enables, for example, relocation of the matrix complex thereon from one site to another, from one culture device to another, and so on.

Compositions of the present disclosure may further comprise a biologically active agent, such as a nutrient, a pharmaceutically active agent, a cell, such as a differentiated cell, such as a blood cell or a chondrocyte, or an undifferentiated cell, such as a stem cell, such as a hematopoietic stem cell or a mesenchymal stem cell, or a nutritional or feeder cell contained within or attached to the functionalized polymer, film or matrix.

The polymer of interest is functionalized. A matrix attached thereto can be functionalized. Moreover, the film can be functionalized, whether the matrix is or not.

Any one or more of the functionalized polymer, film or matrix can carry a biologically active agent, a pharmaceutically active agent or active agent.

Additional features and advantages of the present invention are described in, and will be apparent from the following Detailed Description of the Invention.

### DETAILED DESCRIPTION OF THE INVENTION

The disclosure provides for a film and a matrix, and optionally attached to said matrix, a functionalized biologically compatible polymer, such as a polysaccharide, such as hyaluronate, keratan sulfate and the like, a polypeptide and a polynucleotide.

The term “biologically compatible film, matrix or functionalized polymer” refers to the film matrix or polymer that is a naturally occurring or one that is not toxic to the host. Generally, the metabolites of the film, matrix or functionalized polymer of interest also are not toxic to the host. It is not necessary that any subject composition have a purity of 100% to be deemed biocompatible; indeed, it is only necessary that the subject compositions be non-toxic to the host. Hence, a subject composition may comprise molecules or portions thereof comprising 99%, 98%, 97%, 96%, 95%, 90%, 85%, 80%, 75% or even less of biocompatible molecules.

To determine whether a material is biocompatible, it may be necessary to conduct a toxicity analysis. Such assays are well known in the art. One example of such an assay may be performed with, for example, live carcinoma cells in the following manner: the sample is degraded in 1M NaOH at 37° C. until complete degradation is observed. The solution is then neutralized with 1M HCl. About 200 pL of various concentrations of the degraded sample products are placed in 96-well tissue culture plates and seeded with human carcinoma cells at 10⁴/well density. The degraded sample products are incubated with the cells for 48 hours. The results of the assay may be plotted as % relative growth vs. concentration of degraded sample in the tissue culture well. In addition, reactants, reagents and components of the present film, matrix and functionalized polymer of interest may also be evaluated by well-known in vivo tests, such as subcutaneous implantation in rats to confirm that they do not cause significant levels of irritation or inflammation at the subcutaneous implantation sites. Acceptable levels of toxicity are as known in the art.

The terms “active agent,” “pharmaceutically active agent” and “biologically active agent” are used interchangeably herein to refer to a chemical or biological compound that induces a desired physical, pharmacological or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. As described herein, a biologically active agent includes a living entity, such as a virus, microbe or cell.

The term “target entity” refers to a surface, cell, tissue, organ, cultureware, biological structure, prosthesis, device, medical structure and the like to which a film, matrix or functionalized polymer of interest interacts, reacts or adheres. A “biological surface” is the external, environmentally exposed portion of a biological entity, such as a microbe, virus, cell, tissue, organ and the like, as well as the internal surfaces contained within a structure, such as fenestra, an internal void space, the outer or inner surface of a vessel present within said tissue or organ and so on.

The term “biodegradable” is art-recognized and is intended to indicate that an object degrades during use. In general, degradation attributable to biodegradability involves the degradation of a biodegradable film, matrix or functionalized polymer into oligomers or its component subunits, or digestion, e.g., by a biochemical process, of the matrix, film or functionalized polymer into smaller subunits. In certain embodiments, two different types of biodegradation may generally be identified. For example, one type of biodegradation may involve cleavage of bonds (whether covalent or otherwise) in a component or reactant. In such biodegradation, monomers and oligomers typically result, and even more typically, such biodegradation occurs by cleavage of a bond connecting one or more of the subunits. In contrast, another type of biodegradation may involve cleavage of a bond (whether covalent or otherwise) internal to a side chain or that connects a side chain to a backbone. The side chain may be the functional moiety. For example, a therapeutic agent, biologically active agent or other chemical moiety attached as a side chain to a backbone may be released by biodegradation. In certain embodiments, one or the other or both general types of biodegradation may occur during use of a molecule of interest. As used herein, the term “biodegradation” encompasses both general types of biodegradation as the overall desired function of the molecules of interest wanes.

The degradation rate of a biodegradable structure often depends in part on a variety of factors, including the chemical identity of linkages; the molecular weight, crystallinity, biostability and degree of cross-linking of such molecules; the physical characteristics of the structure, such as the shape and size; the mode and location of administration; and so on. For example, the greater the molecular weight, the higher the degree of crystallinity, and/or the greater the biostability, the biodegradation of any biodegradable molecule is usually slower. The term “biodegradable” is intended to cover materials and processes also termed “bioerodible.” Generally, the rate of degradation is a design choice based on the monomers, functional groups, added ingredients and the like that are used.

In certain embodiments, the biodegradation rate of such molecule may be characterized by the presence of enzymes, for example, a particular protease, lipase, saccharidase and so on. In such circumstances, the biodegradation rate may depend on not only the chemical identity and physical characteristics of the functionalized polymer, film or matrix, but also on the identity, use, presence and the like of any such enzyme.

“Electromagnetic radiation” as used in this specification includes, but is not limited to, radiation having a wavelength of 10⁻²⁰ to 10 meters. Particular embodiments of electromagnetic radiation of the instant invention employ the electromagnetic radiation of: γ radiation (10⁻²⁰ to 10⁻¹³ m), x-ray radiation (10⁻¹¹ to 10⁻⁹ m), ultraviolet light (10 to 400 nm), visible light (400 to 700 nm), infrared radiation (700 nm to 1 mm) and microwave radiation (1 mm to 30 cm).

The term “functionalized” refers to a modification of an existing molecular entity, structure or site to generate or to introduce a new reactive or more reactive group (e.g., acrylate group) that is capable of undergoing reaction with another functional group (e.g., a sulfhydryl group) to form, for example, a covalent bond. For example, carboxylic acid groups can be functionalized by reaction with an acyl halide, e.g., an acyl chloride, again, using known procedures, to provide a new reactive functional group in the form of an anhydride. A functional group is considered equivalent to a reactive group. A substituent can be a functional or reactive group.

The term “film” is used to refer to a biologically compatible structures of the basic form of a skin, membrane, coating, covering, paper, sheet, strip and so on, generally with a two dimensional face substantially greater in size than the width, depth or thickness. The film may be made in situ by gellation, polymerization, solidification, desiccation and the like of a liquid reagent or reagents applied to a site, see, for example, U.S. Pat. Nos. 6,903,199; 6,884,788; and 4,987,893. For example, a film can be made from a polylactic acid or a polyglycolic acid.

The term “hydrogel” is used to refer to a water-swellable polymeric matrix that can absorb water to form gels of varying elasticity, wherein a “matrix” is a three-dimensional network of macromolecules held together by covalent or noncovalent crosslinks. Generally, the network is porous, with pores, network connections and cross linking being variable as a design choice. On placement in an aqueous environment, dry hydrogels swell to the extent allowed by the degree of cross-linking. Alternatively, a hydrogel can be hydrated prior to use. In yet another embodiment, the hydrogel is crosslinked at the point of use. The amount of water absorbed can be controlled by the macromolecule component used. A hydrogel can carry a biologically active agent or a pharmaceutically active agent therein. Procedures for making a hydrogel that entraps and carries an agent are known in the art.

The term “instructional material” or “instructions” includes a publication, a recording, a diagram or any other medium of expression which can be used to communicate the usefulness of a subject composition described herein for a method of treatment or a method of making or using a subject composition. The instructional material may, for example, be affixed to a container which contains the composition or be shipped together with a container which contains the composition or be contained in a kit with the composition or components. Alternatively, the instructional material may be shipped separately from the container with the intention that the instructional material and the composition be used cooperatively by the recipient.

The term “polymer” is used to refer to molecules composed of repeating monomer units, including homopolymers, block copolymers, heteropolymers, random copolymers, graft copolymers and so on. Polymers also include linear polymers as well as branched polymers, with branched polymers including highly branched, dendritic and star polymers. A polymer can be naturally occurring, synthetic or a mixture of both.

A “monomer” is the basic repeating unit in a polymer. A monomer may itself be a monomer or may be dimer or oligomer of at least two different monomers, and each dimer or oligomer is repeated in a polymer.

A “polymerizing initiator” refers to any substance that can initiate polymerization of monomers or polymers by, for example, free radical generation to form a film, matrix or functionalized polymer of interest. The polymerizing initiator often is an oxidizing agent. Exemplary polymerizing initiators include those which are activated by exposure to, for example electromagnetic radiation or heat.

A “methacrylate” refers to a vinylic carboxylate, for example, a methacrylic acid in which the acidic hydrogen has been replaced. Representative methacrylic acids include acrylic, methacrylic, chloroacrylic, cyano acrylic, ethylacrylic, maleic, fumaric, itaconic and half esters of the latter dicarboxylic acids.

It will be understood that “substitution” or “substituted with” includes the implicit proviso that such substitution is in accordance with the permitted valency of the substituted atom and the substituent, and that the substitution results in a stable compound, e.g., which does not spontaneously undergo transformation, such as by rearrangement, cyclization, elimination or other reaction.

The term “substituted” is also contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described hereinabove. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. The invention is not intended to be limited in any manner by the permissible substituents of organic compounds. The term is considered synonymous with functional group and reactive group when the substituent is one that is known, shown or understood to be reactive.

For purposes of this invention, the chemical elements are identified in accordance with the Periodic Table of the Elements, CAS version, Handbook of Chemistry and Physics, 67th Ed., 1986-87, inside cover.

In some embodiments, the disclosure is directed to a functionalized polymer comprising a glycosaminoglycan, mucopolysaccharide, collagen or proteoglycan component, such as hyaluronic acid, heparin sulfate, glucosamines, dermatans, keratans, heparans, hyalurunan, aggrecan and the like, or a saccharide, such as hyaluronic acid, heparin sulfate, keratan sulfate and the like, functionalized by at least one reactive moiety. Those polysaccharides are natural components of extracellular matrices of cells and tissues. However, in general, any biologically compatible polymer can be used as the functionalized polymer, which polymer carries at least one reactive, functional group.

Synthetic polymers that are biocompatible also can be used in the practice of the instant invention. Examples of such synthetic, biocompatible polymers are polyethylene glycol (PEG), polyvinyl alcohol (PVA) and block copolymers, such as the pluronic compounds.

Suitable polymers include biocompatible monomers with recurring units found in poly(phosphoesters), poly(lactides), poly(glycolides), poly(caprolactones), poly(anhydrides), poly(amides), poly(urethanes), poly(esteramides), poly(orthoesters), poly(dioxanones), poly(acetals), poly(ketals), poly(carbonates), poly(orthocarbonates), poly(phosphazenes), poly(hydroxybutyrates), poly(hydroxyl valerates), poly(alkylene oxalates), poly(alkylene succinates), poly(malic acids), poly(amino acids), poly(vinylpyrrolidone), poly(ethylene glycol), poly(hydroxycellulose), chitin and chitosan, and copolymers, terpolymers or combinations or mixtures of the above materials.

Other suitable synthetic polymers include polymers containing amine groups, such as chemically synthesized polypeptides. Such polypeptides may include polynucleophilic polypeptides that have been synthesized to incorporate amino acids containing primary amino groups, for example, lysine and/or amino acids containing thiol groups (such as cysteine). Further suitable synthetic polymers include poly(amino)acids.

A polymer to be functionalized, or monomers thereof, can be obtained from commercial sources, extracted from natural sources using known methods or synthesized from monomers or oligomers, either made or purified as known in the art, or purchased.

A reactive moiety includes any moiety that reacts with a suitable element, chemical group or chemical site on a target entity. One set of target entities are biological structures, such as cells, tissues, organs and the like. Thus, a suitable element, chemical group or chemical site on the surface of a biological structure would be a reactive group found in, for example, a carbohydrate, an amino acid or a nucleic acid, such as an amine group, a carboxylic acid group, a hydroxyl group, a sulfate group and so on. Accordingly, a suitable reactive moiety would be one that reacts with an amine group, a hydroxyl group and so on of the surface of a biological structure.

Other reactive moieties are those which react with elements, chemical groups or chemical sites on biologically compatible materials, such as implants, prostheses, other devices and the like.

A reactive moiety may include alkenyl moieties such as acrylates, methacrylates, dimethacrylates, oligoacrylates, oligomethacrylates, ethacrylates, itaconates or acrylamides. Further reactive moieties include carboxylates and aldehydes. Other reactive moieties may include ethylenically unsaturated monomers including, for example, alkyl esters of acrylic or methacrylic acid such as methyl methacrylate, ethyl methacrylate, butyl methacrylate, ethyl acrylate, butyl acrylate, hexyl acrylate, n-octyl acrylate, lauryl methacrylate, 2-ethylhexyl methacrylate, nonyl acrylate, benzyl methacrylate, the hydroxyalkyl esters of the same acids such as 2-hydroxyethyl acrylate, 2-hydroxyethyl methacrylate, and 2-hydroxypropyl methacrylate, the nitrile and amides of the same acids such as acrylonitrile, methacrylonitrile, methacrylamide, vinyl acetate, vinyl propionate, vinylidene chloride, vinyl chloride, and vinyl aromatic compounds such as styrene, t-butyl styrene and vinyl toluene, dialkyl maleates, dialkyl itaconates, dialkyl methylene malonates, isoprene and butadiene. Suitable ethylenically unsaturated monomers containing carboxylic acid groups include acrylic monomers such as acrylic acid, methacrylic acid, ethacrylic acid, itaconic acid, maleic acid, fumaric acid, monoalkyl itaconate including monomethyl itaconate, monoethyl itaconate, and monobutyl itaconate, monoalkyl maleate including monomethyl maleate, monoethyl maleate, and monobutyl maleate, citraconic acid and styrene carboxylic acid. Suitable polyethylenically unsaturated monomers include butadiene, isoprene, allylmethacrylate, diacrylates of alkyl diols such as butanediol diacrylate and hexanediol diacrylate, divinyl benzene and the like.

In some embodiments, a monomer of a biologically compatible functionalized polymer, film or matrix may be functionalized through one or more thio, carboxylic acid or alcohol moiety located on a monomer of the biopolymer.

The reactive moieties are attached to the functionalized monomer or polymer, film or matrix using known chemistries based on design choice.

Thus, in producing, for example, a functionalized saccharide, a solution comprising the saccharide and a first functional group reactant, such as an alkylene or an acrylate group, are mixed. The solution is stirred, for example, for at least 10 days, at least 11 days or at least 15 days. Alternatively, the solution may be stirred or maintained for about 10 to about 15 days or about 14 to about 15 days. The solution may include a polar solvent, for example an aqueous solvent.

For example, methacrylic anhydride, methacryloyl chloride and glycidyl methacrylate may be used to add methacrylate groups to one or more monomers of a polymer chain. Glycidyl methacrylate may be used, for example, for efficiency of reaction. Further, the modification reagents may be chosen to optimize for a lack of cytotoxic byproducts.

In some embodiments, the number of each of the at least one reactive moiety per polymeric unit may be at least one moiety per about 10 monomeric units, or at least about 2 moieties per about 10 monomeric units. Alternatively, the number of reactive moieties per polymeric unit may be at least one moiety per about 12 monomeric units, or per about 14 monomeric units. For example, there may be at least about one reactive moiety per 15 or more monomeric units. The number of moieties also can range from one per monomer, one per two monomers, one per three monomers, one per 4, 5, 6, 7, 8 or 9 monomers.

Also, the ratio of one of the two reactive moieties to the other can be 5:1, 9:2, 4:1, 7:2, 3:1, 5:2, 2:1, 3:2, 1:1, 2:3, 1:2, 2:5, 1:3, 2:7, 1:4, 2:9 or 1:5 along the full length of the polymer. Preferably, each of the functional moieties is regularly distributed along the length of the polymer and in substantially equal molar amounts. However, the amount of any one reactive moiety type is optimized for reaction with the intended target entity and may result in amounts where the ratio of the two types of reactive moieties deviates from unity.

The functionalized polymer, film or matrix of the invention can also comprise additional biocompatible monomers or polymers so long as there is no interference with the desirable characteristics of the invention. Such additional monomers and polymers may offer even greater flexibility in designing the precise profile desired for, for example, targeted drug delivery, tissue engineering, enhanced administration or the precise rate of biodegradability or biocompatibility desired.

In another embodiment, a method of producing a functionalized polymer or a multiple layer functionalized polymer, film or matrix is provided. A suitable monomer or polymer is exposed to at least one polymerizing initiator whereby producing a polymer or multi-layer polymer, film or matrix of interest. The reactive moiety for polymerizing monomers can also be one of the said at least two different reactive moieties of a polymer of interest.

A polymerization reaction of the present invention can be conducted by conventional methods such as mass polymerization, solution (or homogeneous) polymerization, suspension polymerization, emulsion polymerization, radiation polymerization (using x-ray, electron beam or the like) or the like.

Polymerizing initiators include electromechanical and electromagnetic radiation. Initiation of polymerization may be accomplished by irradiation with light at a wavelength of between about 200 to about 700 nm, or above about 320 nm or higher, or even about 365 nm. In some embodiments, the light intensity is about 4 mW/cm².

Examples of other initiators are organic solvent-soluble initiators such as benzoyl peroxide, azobisisobutyronitrile (AIBN), dibutyl and tertiary butyl peroxide and the like, water soluble initiators such as ammonium persulfate (APS), potassium persulfate, sodium persulfate, sodium thiosulfate and the like, redox-type initiators which are combinations of such initiators and tetramethylethylene, Fe²⁺ salt, sodium hydrogen sulfite or like reducing agent.

Useful photoinitiators are those which can be used to initiate by free radical generation polymerization of monomers with minimal cytotoxicity. In some embodiments, the initiators may work in a short time frame, for example, minutes or seconds. Exemplary dyes for UV or visible light initiation include ethyl eosin 2,2-dimethoxy-2-phenyl acetophenone, 2-methoxy-2-phenylacetophenone, other acetophenone derivatives and camphorquinone. In all cases, crosslinking and polymerization are initiated by a light-activated free-radical polymerization initiator such as 2,2-dimethoxy-2-phenylacetophenone or a combination of ethyl eosin and triethanol amine, for example.

Other photooxidizable and photoreducible dyes that may be used to initiate polymerization include acridine dyes, for example, acriblarine; thiazine dyes, for example, thionine; xanthine dyes, for example, rose bengal; and phenazine dyes, for example, methylene blue. These may be used with cocatalysts such as amines, for example, triethanolamine; sulphur compounds; heterocycles, for example, imidazole; enolates; organometallics; and other compounds, such as N-phenyl glycine. Other initiators include camphorquinones and acetophenone derivatives.

Thermal polymerization initiator systems may also be used. Such systems that are unstable at 37° C. and would initiate free radical polymerization at physiological temperatures include, for example, potassium persulfate, with or without tetramethyl ethylenediamine; benzoylperoxide, with or without triethanolamine; and ammonium persulfate with sodium bisulfite.

A composition of interest comprises a matrix, such as a hydrogel. Any of the known hydrogels or those that can be made as taught herein can be used as a design choice in the instant invention, for example, U.S. Pat. Nos. 6,897,064; 6,872,387; and 6,858,299.

For example, poly(ethylene oxide)-diacrylate (PEODA) may be used, and cross-linked polymer matrices may include cogels of CS-MA (chondroitin sulfate and methacrylate) and PEODA. The CS-MA hydrogels may absorb more water than the PEODA hydrogels, thus, increasing the percentage of CS-MA in the cogels increases the water content.

The mechanical properties of a polymer or a multi-layer polymer, or matrix, such as a scaffold, may also be related to the pore structure. Scaffolds with different mechanical properties are produced depending on the desired clinical application. For example, scaffolds for cartilage tissue engineering in the articular joint must survive higher mechanical stresses than a cartilage tissue engineering system in other body sites. Thus, hydrogels with mechanical properties that are easily manipulated may be desired.

The rheological properties of PEODA and CS-MA are similar and the copolymerization does not alter the properties significantly. Cogels with higher portion of PEODA (100% and 75%) have a higher mechanical strength while cogels with 25% and 0% PEODA exhibit a decrease. The PEODA gels are more highly cross-linked than the CS-MA gel.

Cytotoxicity of the biopolymer scaffold system may be evaluated with any suitable cells, such as fibroblasts, by, for example, using a live-dead fluorescent cell assay and MTT, a compound that actively metabolizing cells convert from yellow to purple, as taught hereinabove.

The matrix can be used with the finalized three-dimensional structure present or a matrix reagent can be treated to adopt the final three-dimensional structure at the site of use, practicing methods known in the art and taught herein.

A functionalized polymer or matrix of interest may contain one or more biologically active agents. The biologically active agent may vary widely with the intended purpose for the composition. The term “active” is art-recognized and refers to any chemical moiety that is a biologically, physiologically, or pharmacologically active substance that acts locally or systemically in a subject. Examples of biologically active agents, that may be referred to as “drugs”, are described in well-known literature references such as the Merck Index, the Physicians Desk Reference and The Pharmacological Basis of Therapeutics, and include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released by the subject composition, for example, into adjacent tissues or fluids on administration to a subject.

Further examples of biologically active agents include, without limitation, enzymes, receptor antagonists or agonists, hormones, growth factors, autogenous bone marrow, antibiotics, antimicrobial agents and antibodies. The term “biologically active agent” is also intended to encompass various cell types and genes that can be incorporated into the compositions of the invention.

In certain embodiments, the subject compositions comprise about 1% to about 75% or more by weight of the total composition, alternatively about 2.5%, 5%, 10%, 20%, 30%, 40%, 50%, 60%, 70% or more, of a biologically active agent.

Non-limiting examples of biologically active agents include following: adrenergic blocking agents, anabolic agents, androgenic steroids, antacids, anti-asthmatic agents, anti-allergenic materials, anti-cholesterolemic and anti-lipid agents, anti-cholinergics and sympathomimetics, anti-coagulants, anti-convulsants, anti-diarrheal, anti-emetics, anti-hypertensive agents, anti-infective agents, anti-inflammatory agents such as steroids, non-steroidal anti-inflammatory agents, anti-malarials, anti-manic agents, anti-nauseants, anti-neoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, benzophenanthridine alkaloids, biologicals, cardioactive agents, cerebral dilators, coronary dilators, decongestants, diuretics, diagnostic agents, erythropoietic agents, estrogens, expectorants, gastrointestinal sedatives, agents, hyperglycemic agents, hypnotics, hypoglycemic agents, ion exchange resins, laxatives, mineral supplements, mitotics, mucolytic agents, growth factors, neuromuscular drugs, nutritional substances, peripheral vasodilators, progestational agents, prostaglandins, psychic energizers, psychotropics, sedatives, stimulants, thyroid and anti-thyroid agents, tranquilizers, uterine relaxants, vitamins, antigenic materials and pro-drugs.

Specific examples of useful biologically active agents the above categories include: (a) anti-neoplastics such as androgen inhibitors, antimetabolites, cytotoxic agents and immunomodulators; (b) anti-tussives such as dextromethorphan, hydrobromide, noscapine, carbetapentane citrate and chlophedianol hydrochloride; (c) antihistamines such as chlorpheniramine phenindamine tartrate, pyrilamine doxylamine succinate and phenyltoloxamine citrate; (d) decongestants such as hydrochloride, phenylpropanolamine hydrochloride, pseudoephedrine hydrochloride and ephedrine; (e) various alkaloids such as codeine phosphate, codeine sulfate and morphine; (f) mineral supplements such as potassium chloride, zinc chloride, calcium carbonate, magnesium oxide and other alkali metal and alkaline earth metal salts; (g) ion exchange resins; (h) antipyretics and analgesics such as acetaminophen, aspirin and ibuprofen; (i) appetite suppressants such as phenyl-propanolamine or caffeine; (j) expectorants such as guaifenesin; (k) antacids such as aluminum hydroxide and magnesium hydroxide; (l) biologicals such as peptides, polypeptides, proteins and amino acids, hormones, interferons, cytokines and other bioactive peptidic compounds, such as calcitonin, ANF, EPO and insulin; (m) anti-infective agents such as anti-fungals, anti-virals, antiseptics and antibiotics; and (n) desensitizing agents and antigenic materials, such as those useful for vaccine applications.

Further, recombinant or cell-derived proteins may be used, such as: recombinant β-glucan; bovine immunoglobulin concentrate; bovine superoxide dismutase; recombinant hirudin (r-Hir), HIV-1 immunogen; recombinant human growth hormone, recombinant EPO (r-EPO); gene-activated EPO (GA-EPO); recombinant human hemoglobin (r-Hb); recombinant human mecasermin (r-IGF-1); recombinant interferon β-la; lenograstim (G-CSF); olanzapine; recombinant thyroid stimulating hormone (r-TSH); topotecan; and any recombinantly produced polypeptide or polynucleotide.

Still further, the following listing of peptides, proteins, and other large molecules may also be used, such as interleukins 1 through 18, including mutants and analogues; interferons, LHRH and analogues, gonadotropin releasing hormone, transforming growth factor (TGF); fibroblast growth factor (FGF); tumor necrosis factor; bone growth factor, nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF-2); bone morphogenetic proteins 1-7 (BMP 1-7); somatostatin; thymosin; superoxide dismutase (SOD); and complement factors, and biologically active analogs, fragments, and derivatives of such factors.

Members of the transforming growth factor (TGF) supergene family, which are multifunctional regulatory proteins, may be incorporated in or on a functionalized polymer or multiple layer polymer, film or matrix of the present invention. Members of the TGF supergene family include the β transforming growth factors (for example, TGF-β1, TGF-β2 and TGF-β3); bone morphogenetic proteins (for example, BMP-1, BMP-2, BMP-3, BMP-4, BMP-5, BMP-6, BMP-7, BMP-8 and BMP-9); heparin-binding growth factors (for example, fibroblast growth factor (FGF), epidermal growth factor (EGF), platelet-derived growth factor (PDGF) and insulin-like growth factor (IGF)), Inhibin A, Inhibin B, growth differentiating factors (for example, GDF-1); and activins (for example, Activin A, Activin B or Activin AB). Growth factors can be isolated from native or natural sources, such as from mammalian cells, or can be prepared synthetically, such as by recombinant DNA techniques or by various chemical processes. In addition, analogs, fragments or derivatives of these factors can be used, provided that they exhibit at least some of the biological activity of the native molecule. For example, analogs can be prepared by expression of genes altered by site-specific mutagenesis or other genetic engineering techniques as known in the art.

Various forms of the biologically active agents may be used. These include, without limitation, such forms as uncharged molecules, molecular complexes, salts, ethers, esters, amides, and the like, which are biologically activated when implanted, injected or otherwise placed into a subject.

In certain embodiments, a polymer of interest can be formed into desired structures, such as films, scaffolds or other three-dimensional structures of interest. In such circumstances, other materials may be incorporated into subject compositions, in addition to one or more biologically active agents. For example, plasticizers and stabilizing agents known in the art may be incorporated in compositions of the present invention. In certain embodiments, additives such as plasticizers and stabilizing agents are selected for their biocompatibility.

A composition of this invention may further contain one or more adjuvant substances, such as fillers, thickening agents or the like. In other embodiments, materials that serve as adjuvants may be associated with the composition. Such additional materials may affect the characteristics of the composition that results. For example, fillers, such as bovine serum albumin (BSA) or mouse serum albumin (MSA), may be associated with the functionalized polymer, film or matrix composition. In certain embodiments, the amount of filler may range from about 0.1 to about 50% or more by weight of the composition, or about percent. Incorporation of such fillers may affect the sustained release rate of any encapsulated substance. Other fillers known to those of skill in the art, such as carbohydrates, sugars, starches, saccharides, celluloses and polysaccharides, including and sucrose, may be used in certain embodiments in the present invention.

Buffers, acids and bases may be incorporated in the compositions to adjust for pH. Agents to increase the diffusion distance of agents released from the composition may also be included.

The charge, lipophilicity or hydrophilicity of any subject composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

Biologically active agents may be incorporated into the functionalized polymer, film or matrix by admixture. Alternatively, the agents may be incorporated into a multi-layer functionalized polymer, film or matrix, or attached to a functionalized polymer, film or matrix of interest by binding these agents to the functional groups on the polymers. Such compositions may include linkages that can be easily biodegraded, for example as a result of enzymatic degradation, resulting in the release of the active agent into the target tissue, where it will exert its desired therapeutic effect.

A simple method for incorporating biologically active agents containing nucleophilic groups into the functionalized polymer, film or matrix involves mixing the active agent with a polyelectrophilic component prior to addition of the polynucleophilic component. By varying the relative molar amounts of the different components of the reactive composition, it is possible to alter the net charge of the resulting polymer, film or matrix composition, for example, to prepare a composition for the delivery of a charged compound, such as a protein or ionizable drug. As such, the delivery of charged proteins or drugs, which would normally diffuse rapidly out of a neutral carrier, can be controlled.

For example, if a molar excess of a component that is polynucleophilic is used, the resulting composition may have a net positive charge and can be used to ionically bind and deliver negatively charged compounds. Examples of negatively charged compounds that can be delivered from these matrices include various drugs, cells, proteins and polysaccharides.

If a molar excess of a component that is polyelectrophilic is used, the resulting composition has a net negative charge and can be used to ionically bind and deliver positively charged compounds. Examples of positively charged compounds that can be delivered from these matrices include various drugs, cells, proteins, and polysaccharides.

A functionalized polymer, film or matrix of the present invention can also be used to maintain various types of living cells or genes. The term “genes” as used herein is intended to encompass genetic material from natural sources, synthetic nucleic acids, DNA, antisense DNA, RNA, siRNA, RNAi and so on.

For example, mesenchymal stem cells can be maintained using the medium of interest. Mesenchymal stem cells may not differentiated and therefore may differentiate to form various types of new cells due to the presence of an active agent or the effects (chemical, physical etc.) of the local tissue environment. Examples of mesenchymal stem cells include osteoblasts, chondrocytes and fibroblasts. For example, osteoblasts can be maintained to produce new bone tissue; chondrocytes can be maintained to produce new cartilage; fibroblasts can be maintained to produce collagen; neurectodermal cells can be maintained to form new nerve tissue; epithelial cells can be maintained to form new epithelial tissues, such as liver, pancreas etc.

The cells or genes may be either allogeneic or xenogeneic in origin. For example, the compositions can be used to co-culture cells or genes from species other than that have been genetically modified. In some embodiments, the compositions of the invention may not easily be degraded in vivo, cells and genes entrapped within the polymer compositions will be isolated from the patient cells and used to proliferate autologous cells.

To entrap the cells or genes within a functionalized polymer, film or matrix, the cells or genes may, for example be pre-mixed with a composition comprising functionalized polymer, and optionally, a further biocompatible polymer. That may occur through a particular reaction or may occur during the making of a multiple layer polymer. Alternatively, the cells may be contained within a target entity attached to a polymer of interest.

The reactive components of the polymer, such as monomers or oligomers, can be infused or instilled at a desired site. The present invention may be prepared to include an appropriate vehicle for this injection, implantation, infusion or direction. Once at the site, the functionalized biologically compatible polymer comprising at least one functional group can be polymerized as taught herein or as known in the art. The polymer then will react with the surface of interest, such as a film or biological surface.

The functionalized polymer, alternatively, may be formed as a solid object, or as a film or mesh that may be used to cover a segment of the area. Known inert ingredients can be mixed with a polymer of interest to make a suitable form, such as film, scaffold, gel and so on, as taught herein.

The film and matrix of interest find use as a culture medium for cells of interest. The film is laid or placed in a suitable culture vessel, such as a Petri dish, a culture bottle and the like. The film can be placed in the vessel and may react with the vessel to become affixed thereto. Some films, such as those based on hyaluronic acid, adhere to certain surfaces. Alternatively, the film can be adhered to the surface using, for example, agar or other adhesive. In another embodiment, the film can be configured to contain reactive groups, as taught herein for a polymer or hydrogel of interest, so that the film is reactive with a surface. Then a matrix, such as a hydrogel, is applied to the surface of the film. The hydrogel can be functionalized so that the hydrogel reacts with the film. The hydrogel can be of varying thickness, as a design choice. A suitable liquid culture medium can be used to make the hydrogel so as to support any cells added to the culture medium of interest.

Often, to more closely simulate the extant environment of a cell in a body, the film/matrix composition of interest can include a membrane-like structure, to provide another or a differentiated surface from the film of interest and the matrix of interest. That membrane-like structure can be obtained using a functionalized polymer of interest. The functionalized polymer can be applied to the surface opposing the film of interest. The functionalized polymer can be added to the matrix or can be applied as a reagent which is gelled or polymerized to form a “membrane” on the surface of the matrix. Cells can be entrapped in the matrix or functionalized polymer or can be seeded in the culture vessel or on the culture medium of interest.

The culture medium of interest can be configured to retain a structure for a prolonged period of time, wherein the liquid medium is replaced periodically, using, for example, color indicators for buffering capacity, the medium can be circulated though the medium on a circulating, continuous basis, and so on. The medium also can be configured to degrade after a period of time, although the three dimensional array provided by the medium of interest can be essential for proper cell proliferation and differentiation, as well as simulating proper tissue development in vitro.

Articular cartilage is a type of hyaline cartilage that lines the surfaces of the opposing bones in a diarthrodial joint. It is an avascular connective tissue responsible for load bearing in synovial joints. Articular cartilage provides a near frictionless articulation between the bones, while also functioning to absorb and to transmit the compressive and shear forces encountered in the joint. Further, since the tissue associated with articular cartilage is aneural, those load absorbing and transmitting functions occur in a painless fashion in a healthy joint.

Unfortunately, cartilage has limited capacity for self-repair. Regardless of the magnitude of trauma and damage that may occur in cartilage, defects do not heal spontaneously. When cartilage tissue is no longer healthy, it can cause debilitating pain in the joint.

Articular cartilage health can be affected by disease, aging, or trauma, all of which primarily involve a breakdown of the matrix consisting of a dense network of proteoglycan aggregates, collagen fibers and other smaller matrix proteins. Cells are unable to induce an adequate healing response because they are unable to migrate to the needed site, being enclosed in lacunae surrounded by a dense matrix. Further, since the tissue is avascular, initiation of healing by circulating cells is limited.

Various surgical techniques and strategies for cartilage repair exist such as abrasion, subchondral drilling, and mosaicplasty. Although they have been used to treat cartilage defects by introducing new cells or tissue to the injury site, those techniques are limited as the repair tissue formed by cell proliferation and differentiation in treated lesions lacks the biological and mechanical properties of native cartilage. The long-term outcome of those techniques has been known to result in mechanically inferior fibrocartilagenous tissue.

Recently introduced tissue engineering methods address the problem of mechanically inferior fibrocartilagenous tissue formation in cartilage defects. Three general strategies for engineering new tissue include utilization of: 1) isolated cells or cell substitutes, 2) tissue-inducing substances and 3) cells placed on or within matrices.

Presently, cell based therapies require harvesting tissue from a donor site, which requires an additional, potentially painful procedure and can lead to morbidity at the donor tissue site. Furthermore, cellular therapies are very costly. Cell banks capable of providing reliable, non-immunogenic, autologous cells are not yet established. Cell transplantation also incurs significant additional costs. Consequently, there is a significant need for technologies capable of harvesting the healing mechanisms of a host in a controlled manner.

The scaffold provided by the medium herein plays a structural role by providing a three-dimensional matrix on which the cells can infiltrate, proliferate, produce matrix, and form functional tissue in a desired shape. They can take the form of solid substrates or softer gels created from synthetic or natural materials. The physical, chemical, and mechanical properties of the scaffold can be tailored for a given clinical application. Furthermore, the scaffold can be designed to present biological signals to further enhance control over cellular functions such as proliferation and differentiation, leading to improved tissue development.

In some embodiments, compositions disclosed herein may be positioned in a surgically created defect that is to be reconstructed, and is to be left in this position after the reconstruction has been carried out. The present invention may be suitable for use with local tissue reconstructions, pedicle flap reconstructions or free flap reconstructions.

For the case of coating an articular surface, at times, injectable materials require the aid of gravity to obtain the desired shape. To enhance the ability to contour defects and control the surface geometry, a composition of interest can be used to better shape hydrogels formed in situ in articular surface defects. Thus, a defect can be treated with a polymer of interest, which functionalized polymer binds to a biological surface. Alternatively, a functionalized polymer is not used. The defect is filled with a matrix, or matrix reagent. Then, the defect is covered with a film of interest. If ungelled or not solidified, the matrix can be solidified or formed by a suitable means depending on the initiator or chemical reaction selected, as a design choice. Thus, for example, a matrix reagent containing a photoinitiator can be gelled by exposing said reactant to a suitable electomagnetic source, which could be exposed to the reagent prior to covering with the film or after the film is placed, said electromagnetic radiation being transmitted through the film. In another embodiment, the defect is covered with a film of interest and then the matrix reagent is instilled in the defect site by a suitable means, such as a syringe, through the film of interest.

The instant invention enables matrix scaffolds that take the desired shape of the defect, promote tissue development by local cells, such as mesenchymal stem cells, and can be implanted by minimally invasive injection, if required, under direct visualization with the help of arthroscopy that is extensively used for the rapid recovery and its minimally invasive nature. Computer assisted navigation technology and fluoroscopy, for example, can be used for the injection of matrix to the target damaged cartilage. Thus, for example, hyaluronic acid based biodegradable films can be used as barriers to contain injected hydrogel within the defect. Hydrogels consist of hydrophilic polymers cross-linked to from a water-swollen, insoluble polymer network. Cross-linking can be initiated by many physical or chemical mechanisms. Photopolymerization is a method to covalently crosslink polymer chains, whereby a photoinitiator and polymer solution (termed “pre-gel” solution) are exposed to a light source specific to the photoinitiator. On activation, the photoinitiator reacts with specific functional groups in the polymer chains, crosslinking them to form the hydrogel.

The reaction is rapid (3-5 minutes) and proceeds at room and body temperature. The procedure could be done on a out-patient basis. Photoinduced gelation enables spatial and temporal control of scaffold formation, permitting shape manipulation after injection and during gelation in vivo. To avoid the gravity forces and to overcome the technical difficulties in surgeries that might be encountered during the injection of matrix to defects that might be in different locations and in various shapes, the biodegradable, for example, transparent films of interest, such as, hyaluronic acid based films, act as barriers to contain the injected material during the solidification (photopolymerization) process. The barriers also are used to prevent adhesion after surgeries and are nonimmunogenic.

Cells and bioactive factors can be easily incorporated into the matrix scaffold by simply mixing with the reagent prior to photogelation. Photopolymerizable materials have been used in a wide variety of biomedical applications, including dentistry, drug delivery, and tissue engineering, and have the potential to create a significant impact in clinical practice.

Whether it is minimally invasive procedure, or arthroscopic procedure or an open procedure, a blend of PEODA (photopolymerizable polyethylene oxide diacrylate) and HA (hyaluronic acid) can be used, along with other hydrogels as a design choice. The HA serves to improve the viscosity, and therefore handling, of the pre-gel solution. It is also a bioactive component that can be used to assist in cell growth and differentiation, such as enhancing stem cells differentiation. On crosslinking of the PEO, the HA is trapped within the hydrogel network, localized to the defect site, and presented to the infiltrating mesenchymal stem cells.

Cartilage poses a particularly difficult challenge for biomaterial integration since it lacks the ability to self-repair and has a dense extracellular matrix that impedes cellular migration and tissue integration. The instant method and materials are used to integrate biomaterials to cartilage that are compatible with a minimally invasive approach. Chondroitin sulphate (CS), a natural cartilage extracellular matrix molecule, was functionalized with aldehyde groups (ALD) and methacrylate groups (MA) to prime the cartilage tissue surface before the hydrogel was injected into the defect. The aldehyde groups reacted with exiting proteins on the cartilage surface. Once the reaction was complete (˜4 min), the pre-gel solution was placed in the defect. Then a film of interest was applied to the defect. On light exposure (6-8 mW/cm², 365 nm UV light), the methacrylate groups in the primer reacted with the same functional groups in the pre-gel solution, resulting in a hydrogel covalently bonded to the cartilage matrix. By modifying the cartilage surface at the defect site, improved integration of biomaterials by direct chemical bonding of the functionalized polymers to the cartilage matrix was obtained. Experiments were performed both in vitro and in vivo in rabbits.

An arthroscopic brush can be used to deliver the functionalized polymer of interest to the tissue surface and flexible soft reamers to ream the surface of the defect without penetrating the tidemark for an enhanced integration and smooth surface. That step is followed by cleansing the by products after the integration is completed. Then, a film of interest, such as a bioabsorbable hyaluronic acid based transparent polymer film, is applied over the defect. This membrane is used to contain the gel in place during injection and ensuing photopolymerization process and to enhance the surface congruity with the neighboring native cartilage.

Drilling into the subchondral bone is a standard practice in orthopedic surgery to improve cartilage repair. Cells from the bone marrow seal off the defect site and form scar tissue. However, the infiltrate often forms a fibrocartilaginous tissue which does not have the mechanical properties of hyaline cartilage and eventually fails. The composition of interest with subchondral drilling procedures offer significant improvements through the prevention of fibrotissue formation. Fibroblasts do not thrive (produce tissue or proliferate) in certain matrices, such as, hydrogels, while bone marrow mesenchymal stem cells (MSCs) and chondrocytes can survive in certain matrices, such as a hydrogel, and form cartilage. Furthermore, in vitro studies have shown that the matrix of interest can be used to reduce formation of the fibrous capsule that often surrounds engineered cartilage. That reduces the risk of fibrocartilage production in the defect site and promotes differentiation of MSCs to form hyaline cartilage.

Therapies for the treatment of osteochondritis dissecans, avascular necrosis and osteoarthiritic degeneration in posttraumatic articular cartilage include, for example, bone marrow-stimulating techniques like abrasion arthroplasty, drilling and microfracturing, new techniques like autologous osteochondral transplantation and autologous chondrocyte transplantation, often with various alternative treatment modalities. Bone marrow stimulating therapy is an inexpensive, low invasive therapy and a good therapeutic option at least for small osteochondritis dissecans lesions and early stages of avascular necrosis. Although autologous chondrocyte transplantation and osteochondral autograft procedures can yield positive 2-year and 4-year results, the long-term results are yet to be defined and other concerns such as the cost, integration of the implant with the grafted side, and fibroblast formation limit use of those methods. The surgical technique, which combines the drilling with the transplantation of newly introduced tissue engineering products, aims to address all the problems and more importantly enables the subchondral bone marrow stimulation that is the key in many orthopaedic diseases. It is an inexpensive, minimally invasive treatment and provides autologous stem cells to the defect surface.

The surgical approach including drilling can be performed in, for example, three different ways:

First approach: drilling is in a retrograde fashion. The defect is located arthroscopically and under direct visualization the base of the defect is penetrated. Then, the drill is forwarded to purchase the bone proximally to the defect. The point from which the drill exits is located far from the defect and joint and preferably in the diaphysis. This point is the location for the entrance point for a cannulated larger diameter drill. That drill use is optional, and used to stimulate the subchondral bone and to deliver the stem cells in the marrow to the defect side. Subchondral bone marrow stimulation is a method of choice in diseases such as osteochondritis dissecans and avascular necrosis and can also be used for cartilage repair in problems such as trauma and osteoarthritis. The drilling follows the use of polymer of interest and can precede the application of the, for example, transparent film onto the defect. The drill is left in place to plug the hole created by drilling through the defect. The tip is visible from the articular surface and the other end reaches the proximal entrance point. Thereby, the cell infiltration is prevented. At that stage, the film is applied. Injection of hydrogel through the film is followed by photopolymerization and removal of the drill to unplug the defect. That is followed by stem cells migration to the defect surface. They seal the defect off and are blended in the hydrogel during and after the photopolymerization process.

Second approach: drilling is in an ante grade fashion. Either the fluoroscopy, or image guided or image free computer assistance guides the drill location. The aim is to penetrate the center of the defect with the guided drill. When the drill purchases the defect base, the bit tip is left as a plug to prevent stem cell migration to the defect. Again over drilling with a cannulated drill is optional. The second approach differs from the first by the use of different surgical aids to locate the defect. The other steps for photopolymerization and injection are the same.

Third approach: microdrilling is performed through the photopolymerized hydrogel. Microdrilling is the final step in the approach.

In some embodiments, the invention contemplates a kit including subject compositions and instructions for use. For example, the kit may comprise a film, which may be functionalized, or film reagent, and matrix, functionalized or not, or matrix reagent, as well as any other necessary reagent, such as an initiator. The kit may contain, for example, a hydrogel, with our without one or more functional groups, or a hydrogel reagent. The kit may further comprise functionalized biologically compatible polymer reagent. The kit may contain suitable instructions.

Contemplated equivalents of the functionalized polymers, matrices, film, subunits and other compositions described herein include such materials which otherwise correspond thereto, and which have the same general properties thereof wherein one or more simple variations of substituents are made which do not adversely affect the efficacy of such molecule or composition to achieve its intended purpose. In general, the compounds of the present invention may be prepared by the methods illustrated in the general reaction schemes as, for example, described above, or by modifications thereof, using readily available starting materials, reagents and conventional synthesis procedures. In the reactions, it is also possible to make use of variants which are in themselves known, but are not mentioned here.

All publications and patents mentioned herein are hereby incorporated by reference in their entirety as if each individual publication or patent was specifically and individually indicated to be incorporated by reference.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. A composition comprising a biologically compatible film and thereon a biologically compatible matrix.

2. The composition of claim 1, wherein said matrix is a hydrogel.

3. The composition of claim 2, wherein said hydrogel comprises poly(ethylene oxide) diacrylate.

4. The composition of claim 3, further comprising a biocompatible polymer.

5. The composition of claim 4, wherein said polymer is hyaluronic acid.

6. The composition of claim 1, wherein said film comprises hyaluronic acid.

7. The composition of claim 1, wherein said film comprises fibrin.

8. The composition of claim 1, wherein said film is obtained by reacting a film reactant to product said film.

9. The composition of claim 1, wherein said matrix is obtained by reacting a matrix reactant to form said matrix.

10. The composition of claim 1, wherein said matrix comprises a functional group reactive with said film.

11. The composition of claim 1, further comprising a biologically compatible functionalized polymer.

12. The composition of claim 11, wherein said functionalized polymer comprises a reactive moiety reactive with said matrix.

13. The composition of claim 12, wherein one said reactive moiety is selected from group consisting of methacrylates, ethacrylates, itaconates and acrylamides.

14. The composition of claim 13, wherein said reactive moiety is methacrylate.

15. The composition of claim 12, wherein said reactive moiety is an aldehyde.

16. The composition of claim 11, wherein said biologically compatible polymer comprises chondroitin sulfate.
